(19) United States
(12) Patent Application Publication
(10) Pub. No.: US 2009/0202444 A1
(43) Pub. Date: Aug. 13, 2009

(54) Treatment and prevention of systemic Xylella fastidiosa infections of plants using antimicrobial metal compositions

(76) Inventor: Karel Newman, Escondido, CA (US)

Correspondence Address: Karel Newman, 3322 San Pasqual Trail, Escondido, CA, 92025-7540, US

(21) Appl. No.: 12/357,310

(22) Filed: Jan. 21, 2009

(51) Int. Cl. A61K 9/12; A61K 33/38; A61K 33/34

(52) U.S. Cl. 424/43; 424/618; 424/630

(57) ABSTRACT

An embodiment of the invention is a treatment for the mitigation and prevention of systemic infection of plants by Xylella fastidiosa using materials derived from bactericidal metals wherein one metal is silver. The candidate bactericidal metal is preferably introduced in metallic, nanocrystalline, salt form, chelated form, or otherwise coupled form. Metal atoms, ions, molecules, or clusters are introduced to the plant to the plant by injection, ballistic insertion, pneumatic insertion, mechanical insertion, manual insertion, root application, aerosolization, or spray in sufficient concentration to treat, cure, and/or prevent plant disease or reduced productivity caused by X. fastidiosa.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND

1. Field

This application relates to antimicrobial metal compositions minimally containing silver in general and specifically used to treat or prevent disease and death in plants caused by Xylella fastidiosa.

### BACKGROUND

2. Prior Art

It has been long known to those of skill in the subject art of this patent that bacteria may reside within plant tissues. Some bacterial agents may reside commensally wherein the organisms consume circulating nutrients, but otherwise do little or no harm to the host plant. However, some bacteria are pathogens invading plants and causing a detrimental systemic infection of cells and tissues. Diseases including Bacterial Wilt, Citrus Variegated Chlorosis, Leaf Scorch, and Pierce's Disease are some examples of injurious and/or fatal plant diseases with bacterial etiologies.

Xylella fastidiosa is a bacterial plant pathogen (Purcell A H, Hopkins D L. Ann. Rev Phytopathol. 1996;34:131-51; California Agricultural Research Priorities: Pierce's Disease (2004) National Research Council of the National Academies. The National Academies Press). The bacterium requires mechanical transfer from an infected host plant to an uninfected host plant to spread. A common mode of transmission is via insect vector, although infection can be transferred by other means including but not limited to contaminated equipment. The bacterium multiplies by colonizing the xylem of dicotyledonous plants and benefits from the nutrient fluids present thereof. Colony growth by the microbe results in the formation of plaques that can eventually occlude the vessel reducing or preventing fluid and nutrient flow. Affected plants may include, but not be limited to, vine crops (eg. Vitis vinifera), fruit crops (eg. Prunus sp.), and ornamental trees (eg. sycamore, oak), and may exhibit initial leaf discoloration followed by leaf necrosis and defoliation as summer heat stress combined with vascular stenosis impacts fluid transfer and tissue hydration. There are a spectrum of tolerances to infection wherein certain plants (eg. Citrus) exhibit moderate tolerance to pathology (eg. chlorosis) whereas other plants variously exhibit significant symptoms and eventually succumb to the infection (eg. V. vinifera grapes). The infection of susceptible plants as with the example of V. vinifera grapes cause the debilitating and, by present belief, invariably lethal Pierce's Disease.

The bacterium X. fastidiosa poses a significant economic impact on both agriculture and horticulture. One may estimate that the lost production and consequential recovery costs per 1000 acres are approximately $3 millions. The viticulture industry has been especially negatively impacted by the disease. During the later years of the 19^{th} century, Pierce's Disease destroyed more than 35,000 acres of grapevines in the Los Angeles basin. Another 50,000 acres of plantings were destroyed in the Napa and San Joaquin valleys during 1935-1940 (Gardner, M. W., Hewitt, W. B. 1974. Pierce's disease of the grapevine: The Anaheim disease and the California vine disease. University of California Press). Up to 50% of plantings in the Temecula growing region were lost in 2001-2003 to Pierce's disease. In the southeastern United States, for example, the occurrence of PD among European wine grapes (Vitis vinifera) was so great and widespread that it always was impractical to grow this crop in states of the Gulf Coastal Plains (Committee on California Agriculture and Natural Resources, National Research Council, 2004). The existence of local reservoirs of the disease within the Rio Grande Valley similarly limits the commercial planting of V. vinifera grapes within the region.

Control of Pierce's Disease in grapes is of high priority in the wine-growing State of California. A cure of X. fastidiosa infections has eluded investigators (California Agricultural Research Priorities: Pierce's Disease (2004) National Research Council of the National Academies. The National Academies Press). The bacterium can be suppressed with antibiotics; however, studies with oxytetracycline have been shown to only afford temporary suppression of disease (D. L. Hopkins, 1979. “Effect of tetracycline antibiotics on Pierce's Disease of grapevine in Florida.” Proc. Fla. State Hort. Soc. 92: 284-285).

Control of bacterial diseases of plants requires costly applications of insecticides to prevent disease transmission, the prompt removal and replacement of infected plants, and adherence to hygienic treatment of work implements. Efforts are in progress to develop disease resistant plants, and will require the eventual replacement of producing plant stocks. Further, the genetic modification of plants may alter characteristics of the parental plant stock. The safe preventative or curative treatment of infected plants could negate the need for replacing valued parental plant varietals and reduce or prevent the need for costly replanting and productivity lags.

It is known by those of skill in the art of this invention that some metals or metal compounds exert an antibacterial affect when contacted with bacteria. These metals tend to assert a broad spectrum antimicrobial activity, and resistance to said activity may be slow to develop (A. B. G. Lansdown. Journal of Wound Care. 11(4): 125-130). For example, topical application of silver nitrate solution to the eyes of neonates is prescribed by law for the prevention of ocular infections and injury associated with Neisseria gonorrhea in addition to other bacterial agents of disease. Topical applications of copper may reduce infections of freshly pruned trees by organisms like Pseudomonas syringae. Thimerosal, a mercurial material, was long used as a bactericidal preservative in vaccines and other medicinal solutions. Bismuth compounds have been demonstrated to assert an antibacterial affect against certain gastrointestinal bacteria. There are other examples known by those with skill in the art and science of microbiology and this document is not intended to present an exhaustive summary of all examples.

Silver is a particularly useful antimicrobial metal in that it asserts a strong antibacterial activity against a broad range of bacterial species while being relatively well tolerated by humans. The phytotoxicity of silver is generally low relative to metal ion concentrations required for inhibition of bacteria. Inhibition of bacterial growth can be attained with concentrations of silver ion below 5 parts per million (ppm) (Appl Environ Microbiol. 2008 April; 74(7): 2171-2178). Certain preparations of other bactericidal metals such as copper can be more likely to be phytotoxic and may require higher concentrations to be effective.

Antibacterial activity of silver ion is, in part, directed against protein sulfhydral groups. Evidence suggests that silver can inactivate the respiratory apparatus of bacteria. Silver ions disrupt electron transfer between cytochrome b and cytochrome d, and flavoprotein-substrate interaction in the NADH and succinate dehydrogenase regions (Biol Metals 1990. 3:151-154). Silver also enhances peroxide-mediated bactericidal activity. Bacterial death rates are interdependent of silver ion concentrations (Appl Environ Microbiol. 2008 April; 74(7): 2171-2178).

There have been various prior disclosures regarding the use of antimicrobial metal-containing compositions including the use of silver to control, prevent, or treat against bacterial infections. The incorporation of antibacterial metals in materials to control microbial growth was disclosed in U.S. Pat. Nos. 4,150,026 and 5,958,440. U.S. Pat. Nos. 6,379,651, 6,426,085 and 6,902,738 describe use of bismuth containing compounds to treat topical oral and digestive system bacterial infections. Burrell and Yin in U.S. Pat. No. 7,008,647 disclosed the use of antimicrobial metals to treat topical acne infections. U.S. Pat. No. 4,559,223 discloses the inhibition of mouth infections and dental caries, plaque formation, gingival destruction and tooth loss by contacting teeth with a composition comprising silver and/or zinc sulfadiazine, and U.S. Pat. No. 6,365,130 disclosed incorporation of antimicrobial metals into chewing gum for control of oral infections. U.S. Pat. Nos. 7,351,684 and 7,462,590 disclose the augmentation of disinfectant peroxides with metals. U.S. Pat. Nos. 7,157,614, 7,179,849, and 7,378,156 pertain to the coating or incorporation of silver into medical devices to provide and sustain an antimicrobial activity, and U.S. Pat. No. 7,348,365 discloses the preparation of nanoparticles for antibacterial preparations. U.S. Pat. Nos. 6,797,743 and 6,905,711 describe the incorporation of antimicrobial metals into polymers to afford an anti bacterial property to said materials, and U.S. Pat. No. 6,509,057 disclosed incorporation of metals into other surface materials. U.S. Pat. No. 6,242,009 discloses the use of chelated metal ions in antibacterial formulations. In the U.S. Pat. No. 6,939,566, Batareseh disclosed the presentation of antibacterial metals minimally containing an organic chelating moiety and useful for disinfection and as a preservative for cut flowers and plants; the disclosure did not provide for a specification as to uses other than treatments for disinfection of topical microorganisms. S. Subramaniam in U.S. Pat. No. 7,381,715 separately disclosed the use of chitosan-chelated silver ions as a vehicle for incorporation of antimicrobial metal into plastics. U.S. Pat. No. 7,354,605 describes methods affecting the controlled release of antimicrobial metal and metal ions incorporated into medical devices. U.S. Pat. No. 7,270,721 discloses the use of silver as an antibacterial and antiseptic in wound dressings. U.S. Pat. No. 6,139,879 discloses chelated metal formulations for the topical prevention and treatment of bacterial disease of plants; the disclosure is a specification for uses of chelated metals whereas the uses teach of applications for topical foliar disease control. None of the cited patents specifically teaches through disclosure the means or uses of any silver metal-containing compounds or materials for the treatment and prevention of systemic bacterial disease of plants.

Copper-based materials have been disclosed in applications with purported benefits in controlling or treating systemic bacterial infections of plants, including infections caused by X. fastidiosa, while exacting minimal phytotoxic side-effects. U.S. Pat. No. 4,544,666 and the related U.S. Patent Application 20060178431 cited the use of injected tannic acid complexes of cupric-ammonium formate as a chemotherapeutic agent against systemic bacterial infections. However, in the 20 years since the issuance of U.S. Pat. No. 4,544,666, diseases including Elm Phloem Necrosis (Elm Yellows) (http://elmyellows.psu.edu/FAQ), Pierce's Disease, and bacterial Citrus Dieback remain classified as incurable.

While the background information illustrated that metals and material derivatives employing silver have been used for topical applications including clinical applications, the application of silver-metal and metal combinations and derivatives to treat and prevent systemic infections of plants has been poorly described. This invention pertains to uses and applications of antimicrobial metals with toxicity against X. fastidiosa, minimal phytotoxicity against infected plants, and negligible potential for toxicity to the human consumers of fruits borne of treated plants.

### SUMMARY

This U.S. Patent communication describes the use and presentation of bactericidal metals and metal compounds containing silver to prevent and treat for plant diseases caused by X. fastidiosa. In accordance with one embodiment, silver-based material in particular, can be used to treat or prevent disease and death in plants caused by Xylella fastidiosa.

### DETAILED DESCRIPTION

Teaching of the invention requires some prerequisite understanding of microbiology, plant biology, and viticulture. Many of the terms and notations have meaning and are generally understood by those of skill in the art from which this invention has been derived.

The term “bacterial infection” in context to this invention pertains to the occupation of subject tissues by bacteria. The subject of this U.S. Patent is the prevention and treatment of plant disease, and accordingly pertains to the infection of plant tissues. While some instances of bacterial residence may produce no deleterious affect or may be beneficial to said tissue, other instances may be damaging or otherwise deleterious to the occupied tissue.

The term “systemic infection” in context to this invention pertains to a widely disseminated occupation of tissues. Microorganisms occupying the cells, intercellular space, and/or vasculature of a plant contribute to a systemic infection.

The term “antimicrobial” in context to this invention pertains to materials that are selectively or preferentially toxic to microorganisms in general and including bacteria in particular. Related terms including “antibacterial” and “bactericidal” in context to this invention pertain to materials that are selectively or preferentially toxic to bacteria.

The term “metals” as used in this invention pertains to atomic elements that classified as metals as outlined in a current periodic table. Metals are electropositive elements, and generally alloy via metallic bonds with other metals, conduct electrical current, can be melted, can be oxidized, and may exhibit a shiny metallic surface in pure form. Elements within the poor metals group may be variously useful, although limitations such as toxicity to humans may reduce candidate materials prepared from the same. Those metals belonging to the alkali metals and alkali base metals group tend to be less useful than metals belonging to the transition metals, wherein the precious metals (eg. gold, silver, and platinum) within the transition metals group may be more generally desirable for antimicrobial applications. Factors such as availability, antimicrobial efficacy, cost, and plant and human toxicity must be considered when selecting a metal for the control and treatment of plant infections as disclosed in this U.S. Patent.

The term “antimicrobial metals” as used in this invention pertains to any individual or combined metals but minimally derived of silver.

The term “ballistic” delivery in context to this invention pertains to any accelerated projectile intended to forcefully insert a solid material as follows. In this U.S. Patent, acceleration of a projectile is achieved by sudden “explosive” propulsion wherein a chemical reaction or other means of sudden creation or release of pressurized gasses results in the transfer kinetic energy. Solid metal, metal particles, pelletized metal compounds, or combinations thereof may be propelled with sufficient velocity so as to imbed deep within a subject plant tissue.

The term “pneumatic” delivery in context to this invention pertains to any accelerated projectile intended to forcefully insert a solid material as follows. In this U.S. Patent, acceleration of a projectile is achieved by sudden impact propulsion wherein a sudden release of pressurized gas results in the transfer kinetic energy. Solid metal, metal particles, pelletized metal compounds, or combinations thereof may be propelled with sufficient velocity so as to imbed deep within a subject plant tissue.

The following examples are disclosed to illustrate the applications and usefulness of the underlying invention.

### EXAMPLE 1

One embodiment in particular employs silver-based material to treat disease caused by Xylella fastidiosa and prevent death in grape vines. Silver carbonate was selected in an initial because of several desirable properties. Properties considered when selecting the material included 1) low relative toxicity to humans, 2) low water solubility (Kₛₚ=6×10⁻¹² M) and accordingly, 3) a theoretical effective silver ion concentration of 25 ppm when present within an aqueous environment, and 4) anticipation of a long residual half-life within the plant tissue through the sustained release of silver ion in accordance with the solubility constant (vv. Kₛₚ). The concentration of silver ion attained with silver carbonate is of importance because the theoretical concentration reached in plant tissue could be several-fold higher than that reported to assert an antimicrobial concentration in in vitro studies.

### EXAMPLE 2

Silver carbonate was mixed with water. 2 grams of the metal salt were added to 200 milliliters (mL) of deionized water. While stirring, 20 mL of the suspension was drawn into ChemJet® Injectors. Individual holes were bored to a depth of approximately ½ inch placed approximately 6 inches below the main cordon branch point of Xylella fastidiosa infected subject grapevines. Foil wrapped injectors containing silver carbonate slurry were installed on individual vines, and the injection plungers were deployed.

### EXAMPLE 3

Individual grape vines were monitored during the study period. Branch samplings were variously collected for later examination including analysis for residual X. fastidiosa nucleic acid by the polymerase chain-reaction method (PCR). Reduced detectible X. fastidiosa nucleic acid levels in analyzed plant tissue following treatment indicate effective control against the infection. Individual vines responded variously to treatment. Pierce's Disease infected and symptomatic Vitis vinifera var. Zinfandel vine recovered and exhibited vigorous growth of new vines near the cordon branch points. The nascent canes grew up to 4 feet in length within 17 weeks following treatment, and exhibited a delayed fruiting cycle.

### EXAMPLE 4

Silver nanoparticles can be selected because of several desirable properties. Properties considered when selecting the material included 1) low toxicity to humans, 2) low ionic release thereby supporting continued release of metal ions, 3) a size than can support transport through the vasculature of the vine, and 4) anticipation of a long residual half-life within the plant tissue.

### EXAMPLE 5

Silver nanoparticles can be suspended in an aqueous solution. An amount of 1 microgram to 10 grams of the microcrystalline metal, and preferably 1 milligram can be added per 200 milliliters (mL) of deionized water. The suspension may be injected in a manner like that described in example 2, or separately sprayed onto emergent or emerged leaves of grape vines.

### EXAMPLE 6

Silver and copper nanoparticles are combined in 1 part to 10 parts proportion as determined by molecular weight. An amount of 1 microgram to 10 grams of the material, and preferably 10 milligrams can be added per 200 milliliters (mL) of deionized water. The suspension is drawn into ChemJet® Injectors in volumes up to 20 mL. The suspension may be injected in a manner like that described in example 2.

### EXAMPLE 7

Bismuth bitartrate, a metal-based medication with a demonstrable antibacterial activity, can be prepared in combination with silver particles as an aqueous suspension. An amount of 1 microgram to 100 grams of the combined material may be added per 200 milliliters (mL) of deionized water. The suspension can be drawn into ChemJet® Injectors in volumes up to 20 mL. The suspension may be injected in a manner like that described in example 2.

### EXAMPLE 8

Antimicrobial metals, metal fragments, metal particles, and metal salts may be introduced into plant tissues by impact delivery. One approach envisions using 1 or more materials formulated to create a deliverable projectile for optimal penetration and distribution of kinetically propelled residuals. Another approach envisions delivery as solid metal spikes that may or may not be coated with a second material. Impact delivery may be produced with a hammer or other methods known in the art.

### EXAMPLE 9

Antimicrobial metals, metal fragments, metal particles, and metal salts may be introduced into X. fastidiosa infected plant tissues by pneumatic delivery. One approach envisions using 1 or more materials to create a deliverable projectile for optimal penetration and distribution of pneumatically propelled residuals. Another approach envisions delivery as solid metal spikes that may or may not be coated with a second material. Pneumatic delivery may be produced with an air gun or other methods known in the art.

### EXAMPLE 9

Antimicrobial metals, metal fragments, metal particles, or metal salts may be introduced into X. fastidiosa infected oak trees by any feasible method. One approach employs 1 or more materials to create a deliverable projectile for optimal penetration and distribution of propelled residuals. Another approach envisions impact delivery of solid metal spikes that may or may not be concurrently administered with one or more additional materials.

What is claimed is:

1. A method for treating plants comprising introduction of or exposure to a therapeutically effective amount of one or more antimicrobial metals to treat or prevent an infection with Xylella fastidiosa wherein the one or more antimicrobial metals in contact with plant tissue, or introduced as a solid or fluid, releases atoms, ions, molecules, or clusters wherein at least one antimicrobial metal is silver and wherein the treatment is at a concentration sufficient to inhibit growth of the microorganism.

2. A method according to claim 1 wherein the at least one antimicrobial metal is in metallic, nanocrystalline, salted, chelated, alloyed, or otherwise chemically coupled form or formulated with an inert carrier, and preferably introduced as a solid, or liquid suspension, or solution.

3. A method according to claim 1 wherein at least one antimicrobial metal is introduced by injection, ballistic insertion, pneumatic insertion, mechanical insertion, manual insertion, root application, aerosolization, or spray.
